SEQUENCE LISTING (4 sequences)

SEQ ID NO: 1 - DNA, 372 bp, Lama glama
caggtaaagctggaggagtctgggggaggactggtgcaggttggggactctctgagactctcctgtgcagcctccggacgcaccttcagtaactataccatggcctggttccgccagtttccagggaaggagcgtgagtttgtagcagtagttagtcgggggggtggcgccacagactatgcagactccgtgaagggccgattcaccatctccagagacaacgccaagaacaccatgtatctgcaaatgaacagcctgaaaactgaggacacggccgtctattactgtgcagcgggtacagacctaagttactattacagcacaaaaaaatgggcctactggggccaggggacccaggtcaccgtctcctca

SEQ ID NO: 2 - protein, 124 aa, Lama glama
QVKLEESGGGLVQVGDSLRLSCAASGRTFSNYTMAWFRQFPGKEREFVAVVSRGGGATDYADSVKGRFTISRDNAKNTMYLQMNSLKTEDTAVYYCAAGTDLSYYYSTKKWAYWGQGTQVTVSS

SEQ ID NO: 3 - protein, 21 aa, Escherichia coli
MKKTAIAIAVALAGFATVAQA

SEQ ID NO: 4 - protein, 21 aa, Artificial Sequence
  Linker
GQAGQGSGGGSGGGSGGGGGS

(19) United States
(12) Patent Application Publication
(10) Pub. No.: US 2013/0129727 A1
(43) Pub. Date: May 23, 2013

(54) METHODS AND SYSTEMS FOR INCREASING PROTEIN STABILITY

(71) Applicant: Nanjingjinsirui Science & Technology Biology Corp, Nanjing (CN)

(72) Inventors: Jianbing ZHANG, Orleans (CA); Shu WU, Nanjing (CN); Jieying LIU, Beijing (CN)

(73) Assignee: Nanjingjinsirui Science & Technology Biology Corporation, Nanjing (CN)

(21) Appl. No.: 13/680,523

(22) Filed: Nov. 19, 2012

Related U.S. Application Data
(60) Provisional application No. 61/561,052, filed on Nov. 17, 2011.

(51) Int. Cl. C07K 19/00

(52) U.S. Cl. CPC C07K 19/00
USPC 424/134.1; 530/387.3; 435/69.6; 435/7.92; 435/252.33

(57) ABSTRACT

Methods and systems for increasing stability of a target polypeptide in a serum are described. The methods and systems utilize a fusion protein comprising a single-domain antibody against a serum albumin (SASA), the target polypeptide and optionally a linker. The fusion protein has a significantly prolonged serum half life in comparison with the target polypeptide alone. The SASA fusion tag also facilitates the expression and purification of the fusion protein. This allows direct in vivo screening or utilization of the target polypeptide for its biological activity or efficacy regardless of its intrinsic serum half life, which has significantly increased the number of candidates for the development of novel protein based diagnosis or treatment.

### CROSS-REFERENCE TO RELATED APPLICATIONS

This application claims priority under 35 U.S.C. §119 to Provisional Patent Application No. 61/561,052, filed on Nov. 17, 2011, the disclosure of which is incorporated by reference herein in its entireties.

### BACKGROUND OF THE INVENTION

The development of therapeutic proteins and structural studies call for easy and more efficient production of purified functional proteins. Recently, a number of methods have been established for screening protein expression and affinity to targets, protein expression, purification and further engineering for in vivo testing [1, 2, 3, 4, 5]. Several expression and purification systems have been developed using fusion tags, such as His-tag [6, 7], glutathione S-transferase (GST) [8, 9, 10], maltose-binding protein (MBP) [11, 12], chintin-binding domain [13], Strep-tag [14, 15, 16] and FLAG-tag [17, 18]. Evaluated by factors such as expression level and solubility of the fusion protein, specificity and affinity of the purification chromatography, material costs and time requested, these systems have their advantages and shortcomings. For instance, His-tag provides good yield using inexpensive resin, but often results in moderate protein purity. Epitope-based tags such as FLAG can achieve high purity, but the approach suffers costly resin and low capacity [19].

Protein stability is another common problem in protein expression, purification, formulation, and storage. None of the above mentioned fusion tags is known to have the ability to extend the half lives of proteins in serum, which is crucial for in vivo testing of the selected target proteins. Antibodies, vaccines, hormones, growth factors, ligands and other proteins have been developed and used as diagnostic or therapeutic proteins. Many of these proteins, especially small ones [20, 21], often have a short serum half life ranging from minutes to approximately one hour. Several strategies have been developed to avoid the clearance of protein drugs from circulation. The widely used approaches for extending the half lives of short-lived molecules are conjugation with a chemical attachment such as polyethylene glycol (PEG) [22, 23], or fusion with a protein having a long serum half life, such as serum albumin [24, 25, 26] or the Fc region of an antibody [27, 28]. Unfortunately, these conjugated macromolecules are relatively large and complicated for large-scale expression, not desirable for in vivo screening. In addition, the conjugation step is usually performed subsequent to initial purification of the protein, which adds time and costs to the whole process.

A fast screening system for expression, biophysical-properties and affinity (FASEBA) analysis involving a protein anchor that comprises an antibody or antibody fragment has been reported. See WO/2011/020183 and [30], the disclosure of each of which is incorporated herein by reference. However, the utility of the protein anchor in increasing protein stability in serum has not been reported.

There is a need for new methods and systems for producing proteins with improved protein stability in serum, which are efficient and costs effective from the selection of a target protein, to the expression and purification of the selected target protein, and to the subsequent animal testing of the target protein. Such methods would facilitate the development of novel protein-based diagnosis or treatment. Embodiments of the present invention relate to such methods.

### BRIEF SUMMARY OF THE INVENTION

It has been discovered in the present invention that a single-domain antibody against serum albumin (SASA) can be used as a fusion tag to facilitate the expression and purification of a fusion protein comprising a target polypeptide and the SASA. In particular, it has been surprisingly discovered that the fusion protein has significantly prolonged stability as compared to the target protein alone in serum or a composition containing a serum albumin.

Accordingly, one general aspect of the present invention relates to a method of increasing the stability of a target polypeptide in a serum, the method comprising:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) administering a composition comprising the isolated fusion protein to the serum, wherein the fusion protein has a longer half-life than the target polypeptide alone in the serum.

Another general aspect of the present invention relates to a method of increasing the stability of a target polypeptide in a composition, the method comprising:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) combining the isolated fusion protein with the serum albumin in the composition, wherein the fusion protein has a longer half-life than the target polypeptide alone in the composition.

In a preferred embodiment of the present invention, the fusion protein comprises an sdAb having the amino acid sequence of SEQ ID NO: 2, SEQ ID NO: 6 or SEQ ID NO: 8.

In a particular embodiment, the present invention relates to a method of obtaining an isolated fusion protein having increased serum stability, the method comprising:
(a) expressing the fusion protein by a recombinant cell, wherein the fusion protein comprises a fusion tag comprising the amino acid sequence of SEQ ID NO: 2, 6 or 8, the target polypeptide and an optional linker from a recombinant cell, wherein the fusion tag is fused to the carboxyl-terminus or amino-terminus of the target polypeptide, and the optional linker separates the fusion tag and the carboxyl-terminus or amino-terminus of the target polypeptide;
(b) obtaining at least one of the lysate, periplasmic extract and supernatant of the recombinant cell comprising the fusion protein;
(c) applying the at least one of the lysate, periplasmic extract and supernatant to a serum albumin affinity chromatography column; and
(d) isolating the fusion protein from the column, wherein the fusion protein has a longer half-life than the target polypeptide alone in a serum.

In a preferred embodiment of the present invention, the target polypeptide is an antibody, vaccine, hormone, growth factor, protein ligand, or any other polypeptide that has been or will be developed or used for a therapeutic or diagnostic purpose, or a protein subject to structural and/or functional analysis.

Another general aspect of the present invention relates to a system for increasing the stability of a target polypeptide, comprising:
(a) a recombinant cell for expressing a fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide;
(b) a solid support for capturing the fusion protein via specific binding between the sdAb in the fusion protein and the serum albumin associated with the solid support;
(c) a buffer having a pH of 1 to 4 or 9 to 14 for eluting the captured fusion protein from the solid support to obtain an isolated fusion protein; and
(d) a composition comprising the serum albumin for combining with the isolated fusion protein to increase to the stability of the fusion protein.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is schematic diagram of pMustKey and pMustKey-N vectors: (A) the pMustKey vector, SASA-tag and His-tag were fused to the C-terminus of the target protein; and (B) the pMustKey-N vector, SASA-tag and His-tag were fused to the N-terminus and C-terminus of the target protein, respectively, wherein SP: ompA signal peptide, MKKTAIAIAVALAGFATVAQA (SEQ ID NO:3); Linker 1: GQAGQGSGGGSGGGSGGGGGS (SEQ ID NO:4); and Linker 2: GLN;

FIG. 2 shows the expression levels of eleven Fabs and Fab-SASA fusion proteins evaluated by Western blot, the same amount of cell lysates of each Fab and Fab-SASA fusion protein was loaded on 12% SDS-PAGE;

FIG. 3 shows photos of SDS-PAGE of Fab-SASA fusion protein and Fab purified by Ni affinity chromatography and BSA-conjugated affinity chromatography: (A) crude and purified protein fractions from BSA-column and Ni-NTA column: Lane 1: MW marker; Lane 2: supernatants of cell lysates; Lane 3: BSA-column flow-through; Lane 4: eluate from BSA-column; Lane 5: supernatant of cell lysates; Lane 6: Ni-NTA column flow-through; Lane 7: wash from Ni-NTA column; Lane 8: eluate from Ni-NTA column; (B) purified Fab-SASA fusion by Ni magnetic beads and BSA magnetic beads: Lane 1: MW marker; Lanes 2 and 3: Ni magnetic beads purified Fab-SASA fusion; Lanes 4 and 5: Fab-SASA fusion purified using BSA magnetic beads from the same amount of cell lysate; and

FIG. 4 illustrates concentrations of Fab-SASA fusion protein and Fab at various time points after the proteins were intravenously (i.v.) injected into mice separately.

### DETAILED DESCRIPTION OF THE INVENTION

Various publications are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. It must be noted that as used herein and in the appended claims, the singular forms “a,” “an,” and “the” include plural reference unless the context clearly dictates otherwise.

Certain terminology is used in the following description for convenience only and is not limiting. Unless defined otherwise, all technical and scientific terms used herein have the same meaning as is commonly understood by one of skill in the art to which this invention belongs.

As used herein, a “fusion tag” is a polypeptide sequence that can be operably linked to a target protein or polypeptide to generate a fusion protein for the ease of subsequent manipulation, such as for the expression, purification, in vitro and in vivo analysis and characterization of the protein, or diagnostic or therapeutic application. A fusion tag may exhibit one or more properties. For example, the fusion tag may selectively bind to a purification medium that contains a binding partner for the fusion tag and allows the operably linked target polypeptide to be easily purified. A fusion tag may be a ligand that binds to a cellular receptor, the interaction of which will allow a target polypeptide that is operably linked to the fusion tag to be specifically targeted to a specific cell type based on the receptor expressed by the cell. The fusion tag may also be a polypeptide that serves to label the operably linked target polypeptide. Examples of fusion tags include, but are not limited to, single-domain antibody (sdAb) that binds specifically to a serum albumin (SASA), glutathione-S-transferase (GST), maltose binding protein (MBP), avidin, biotin, or streptavidin (Strep-tag), polyhistidine (His-tag), chintin-binding domain, FLAG-tag, a ligand of a cellular receptor, serum albumin, the Fc region of an antibody, green fluorescent protein (GFP), red fluorescent protein, yellow fluorescent protein, cayenne fluorescent protein, etc.

As used herein, “single-domain antibody” or “sdAb” refers to the antigen-binding site of a heavy-chain antibody (HCAb) of camelids, such as camel, llama and alpaca and sharks, which is naturally devoid of light chains. The antigen-binding site of HCAb of camelids is formed only by a single variable domain designated VHH or VNAR. The sdAbs usually exist as monomeric proteins having relatively small sizes. See Wesolowski et al., Med Microbiol Immunol (2009) 198:157-174.

As used herein, “SASA”, “single-domain antibody against a serum albumin,” “sdAb against a serum albumin” and “single-domain antibody (sdAb) that binds specifically to a serum albumin” shall all have the same meaning, referring to a single-domain antibody that binds specifically to a serum albumin, the most abundant protein in animal blood plasma. Examples of SASA include, but are not limited to, sdAbs against human serum albumin or sdAbs against bovine serum albumin (BSA), see e.g., [29] and WO 2010/043057, the disclosure of each of which is incorporated herein by reference.

As used herein, “binds specifically to” or “against” when used in connection with an sdAb and an albumin refers to the antibody-antigen binding or interaction between the sdAb and the albumin. An sdAb can bind to an albumin with a dissociation constant (K_{D}) of 10⁻⁷ to 10⁻¹². Specific antibody-antigen binding can be determined in any suitable manner, including, for example, scatchard analysis and/or competitive binding assays, such as radioimmunoassays (RIA), enzyme immunoassays (EIA) and sandwich competition assays, and the different variants thereof known in the art; as well as the other techniques mentioned herein. Methods for determining the binding affinities or dissociation constants are known to those skilled in the art, such as the techniques described in [29], which are incorporated herein by reference.

As used herein, the “half-life” of a polypeptide refers to the time taken for the concentration of the polypeptide to be reduced by 50% in an assay conducted in vivo or in vitro. The reduction can be caused by degradation, clearance or sequestration of the polypeptide in the assay. The half-life of a polypeptide can be determined in any manner known in the art in view of the present disclosure, such as by pharmacokinetic analysis. For example, to measure the half-life of a polypeptide in vivo, a suitable dose of the polypeptide is administered to a warm-blooded animal (i.e. to a human or to another suitable mammal, such as a mouse, rabbit, rat, pig, dog or a primate); blood samples or other samples from said animal are collected; the level or concentration of the polypeptide in the blood sample is determined; the time until the level or concentration of the polypeptide has been reduced by 50% compared to the initial level upon dosing is calculated based on the measured data. See, e.g., Kenneth, A et al: Chemical Stability of Pharmaceuticals: A Handbook for Pharmacists and Peters et al, Pharmacokinete analysis: A Practical Approach (1996).

As used herein, an “increase in half-life” refers to an increase in any one of the parameters used to describe the protein half-life, such as the t½-alpha, t½-beta and the area under the curve (AUC), any two of these parameters, or essentially all of these parameters.

An expression and purification system that uses SASA as a fusion tag is described in the present application. The system eases the screening of protein candidates, subsequent expression and purification of the target proteins. It also makes it easy for adaption of the purified proteins for animal testing, or diagnostic or therapeutic applications. It offers a rapid and simple solution for the production of functional proteins that can be employed in various applications, such as therapeutic protein development and protein engineering. In particular, it was surprisingly discovered that, a SASA fusion tag stabilizes a target protein in certain environment, such as a serum or a composition containing a serum albumin. A fusion protein containing a target polypeptide and a SASA fusion tag has a longer half life than the target polypeptide alone in the serum or the composition. This allows direct in vivo screening or utilization of the target polypeptide for its biological activity or efficacy regardless of its intrinsic serum half life, which has significantly increased the number of candidates for the development of novel protein based diagnosis or treatment.

In one general aspect, the present invention relates to a method of increasing the stability of a target polypeptide in a serum. The method comprises:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) administering a composition comprising the isolated fusion protein to the serum, wherein the fusion protein has a longer half-life than the target polypeptide alone in the serum.

Preferably, a method according to an embodiment of the present invention utilizes the SASA fusion tag to facilitate protein expression and purification, thus the method further comprises:
(a) expressing the fusion protein by a recombinant cell;
(b) obtaining at least one of the lysate, periplasmic extract and supernatant of the recombinant cell comprising the fusion protein;
(c) applying the at least one of the lysate, periplasmic extract and supernatant to a solid support to capture the fusion protein via specific binding between the sdAb in the fusion protein and the serum albumin associated with the solid support; and
(d) eluting the captured fusion protein from the solid support to obtain the isolated fusion protein.

According to embodiments of the present invention, the target polypeptide can be any protein of interest. Preferably, the target polypeptide has been or will be developed or used for a therapeutic or diagnostic purpose. Examples of such target polypeptides include, but are not limited to, an antibody, vaccine, hormone, growth factor, protein ligand, etc. The target polypeptide can also be proteins subject to structural and/or functional analysis.

According to embodiments of the present invention, any single-domain antibody that binds specifically to a serum albumin can be used as a fusion tag in the present invention.

In a preferred embodiment, the sdAb comprises a camelid VHH that binds specifically to a human serum albumin (HSA) or bovine serum albumin (BSA), such as BSA 8, BSA 12 and BSA 16 (see [29] and WO 2010/043057).

In a particular embodiment, the SASA comprises the amino acid sequence of BSA 12 (SEQ ID NO: 2). The SASA can be encoded by a polynucleotide sequence comprising SEQ ID NO: 1:

In another embodiment, the SASA comprises the amino acid sequence of BSA 8 (SEQ ID NO: 6). The SASA can be encoded by a polynucleotide sequence comprising SEQ ID NO: 5:

In yet another particular embodiment, the SASA comprises the amino acid sequence of BSA 16 (SEQ ID NO: 8). The SASA can be encoded by a polynucleotide sequence comprising SEQ ID NO: 7.

In an embodiment of the present invention, the fusion protein comprises a second fusion tag at the amino-terminus or carboxyl-terminus of the fusion protein. The second fusion tag can be, for example, GST, MBP, His-tag, etc.

The fusion protein according to embodiments of the present invention can be used for various purposes in view of the present disclosure. For example, it can be used for drug screening or target identification purposes, e.g., via assaying the affinity of the target protein to a binding partner, etc. It can also be used in a diagnostic method, particularly if the method involves administering the target protein to the serum. It can further be used for therapeutic purpose, particularly if the target protein is known to be unstable in the serum.

Thus in the administering step according to embodiments of the present invention, a composition comprising the isolated fusion protein can be administered to the serum in vivo or in vitro for any purpose. For example, a composition comprising the fusion protein can be administered to the serum in vivo in a subject in need of a diagnosis or treatment involving the target protein. The fusion protein can also be administered to a sample of serum in vitro for screening purpose. The sdAb in the fusion protein binds specifically to an albumin in the serum in vivo in the subject or in vitro in a serum sample.

Preferably, the sdAb fusion tag does not interfere with the function of the target polypeptide, and the fusion protein has the same or similar therapeutic and/or diagnostic property as the target polypeptide alone.

According to an embodiment of the present invention, the fusion protein is made by a recombinant cell that is transformed with a gene encoding the fusion protein.

Expression vectors encoding the fusion protein and recombinant cells expressing the fusion protein can be constructed using methods known in the art in view of the present disclosure.

In one embodiment, the expression vector comprises a first polynucleotide sequence encoding the sdAb linked in frame to the 5′-end or 3′-end of a second polynucleotide sequence encoding the target polypeptide, optionally separated by a third polynucleotide sequence encoding a linker.

In a preferred embodiment, the first polynucleotide encodes a camelid VHH that binds specifically to a bovine serum albumin (BSA), such as a polynucleotide sequence encoding the amino acid sequence of SEQ ID NO: 2, 6 or 8, or a polynucleotide sequence comprising SEQ ID NO: 1, 5 or 7.

The expression vector may further comprise a fourth polynucleotide sequence encoding a signal peptide linked in frame to the 5′-end of the coding sequence of the fusion protein to allow secretion of the fusion protein to the supernatant of a recombinant cell.

A recombinant cell expressing the fusion protein according to the present invention can be made, for example, by transforming a host cell with an expression vector using methods known in the art. The host cell can be any cell suitable for recombinant production of the fusion protein, such as a mammalian cell, a plant cell, a yeast cell, a bacterial cell, etc.

In a preferred embodiment, the recombinant cell is an Escherichia coli cell.

According to an embodiment of the present invention, a fusion protein produced by the recombinant cell can be isolated or purified using methods known in the art, such as those described in WO/2011/020183, which is incorporated herein by reference in its entirety. For example, a fusion protein according to the present invention can be expressed by the recombinant cell as a cytoplasmic protein, a periplasmic protein or a secreted protein. A cell lysate, periplasmic extract or supernatant comprising the fusion protein can be obtained using methods known in the art in view of the present disclosure. The fusion protein can be isolated from the lysate, periplasmic extract or supernatant.

In a particular embodiment of the present invention, the fusion protein is isolated by applying the cell lysate, periplasmic extract or supernatant to a solid support associated with an albumin that binds specifically to the sdAb. The solid support can be, for example, a column packed with resins coated with or conjugated to the albumin, such as a serum albumin affinity chromatography column. The fusion protein is captured to the solid support via specific binding between the sdAb and the albumin. The captured fusion protein is then eluted from the solid support under conditions where the interaction between the sdAb and the albumin is interrupted.

In a particular embodiment, the fusion protein is eluted from the solid support with a buffer at a low or high pH, such as a pH of about 1.0 to about 4.0, or about 9 to about 14. Preferably, the fusion protein is eluted at a pH of about 1.0, 2.0, 3.0, 4.0, 9.0, 10.0, 11.0, 12.0, 13, 0 or 14.0.

In view of the present disclosure, the stability of the protein can be measured by the half life of the protein using methods known in the art, such as pulse-chase analysis and cycloheximide blocking. See, e.g., Zhou, Methods Mol Biol. 2004; 284:67-77.

The fusion protein according to an embodiment of the present invention has at least the features of a high affinity (dissociation constant <10 nM) to the albumin and increased protein stability in serum or a composition containing serum albumin.

Serum albumin has been used as an inert carrier protein to prolong the serum half-life of a large number of proteins [32], mainly because it is the most abundant protein in blood plasma and has a long serum half life of 19 days in humans [33]. However, its presence in a serum does not guarantee protein stability as evident by the short serum half life ranging from minutes to approximately one hour for many proteins, particularly the small ones. Albumin-binding moieties were reported for extending serum half life of therapeutic proteins [21, 34, 35, 36]. However, the mechanisms of action remain unclear.

It is discovered in the present invention that, SASA, an albumin-binding moiety that is structurally and functional different from those used in the prior reports, increased the stability of a target polypeptide fused to the SASA in serum. Without wishing to be bound by theory, it is believed that specific binding between the sdAb in the fusion and the albumin in the serum contributes to the increased stability. Fusion proteins obtained by methods of the present invention have prolonged stability in serum as well as a composition containing a serum albumin.

Thus, another general aspect of the present invention relates to a method of increasing the stability of a target polypeptide in a composition, the method comprising:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) combining the isolated fusion protein with the serum albumin in the composition, wherein the fusion protein has a longer half-life than the target polypeptide alone in the composition.

In one embodiment of the present invention, the method further comprises administering the composition to a subject in need of a diagnosis or treatment involving the target polypeptide.

In another embodiment of the present invention, the method further comprises administering the composition to the serum in vivo or in vitro for identifying a diagnostic or therapeutic agent.

The target polypeptide can be an antibody, vaccine, hormone, growth factor, protein ligand, or any other polypeptide that has been or will be developed or used for a therapeutic or diagnostic purpose, or a protein subject to structural and/or functional analysis.

Another general aspect of the present invention relates to a system for increasing the stability of a target polypeptide, comprising:
(a) a recombinant cell for expressing a fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide;
(b) a solid support for capturing the fusion protein via specific binding between the sdAb in the fusion protein and the serum albumin associated with the solid support;
(c) a buffer having a pH of 1 to 4 or 9 to 14 for eluting the captured fusion protein from the solid support to obtain an isolated fusion protein; and
(d) a composition comprising the serum albumin for combining with the isolated fusion protein to increase to the stability of the fusion protein.

The following specific examples of the invention are further illustrative of the nature of the invention, it needs to be understood that the invention is not limited thereto.

### Example

Materials and Methods

Construction of Expression Vectors

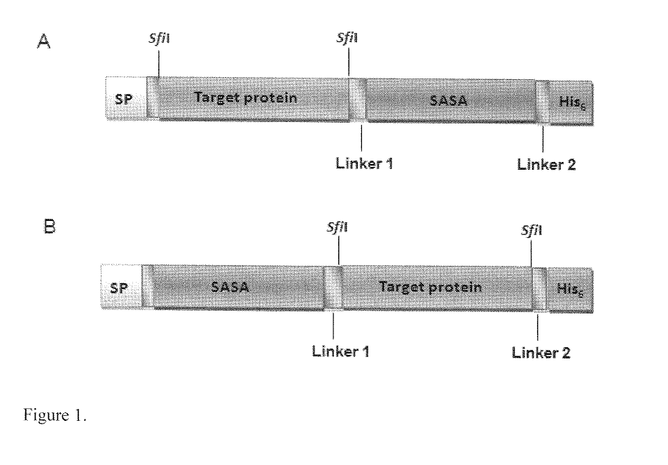

pMustKey was constructed by inserting DNA encoding SASA in an E. coli expression vector pSJF2 [31] (FIG. 1).

DNA encoding 11 randomly selected Fabs (antigen-binding fragments) were PCR amplified and inserted into pMustKey vector using sfiI restriction sites on both ends of the Fab fragments. The 11 Fab genes were also inserted into pSJF2 vector, which did not include SASA. The constructs containing the coding regions of the Fabs and Fab-SASA fusions were confirmed by DNA sequence analysis.

Expression of Proteins and Protein-SASA Fusions

The expression vectors harboring each Fab and Fab-SASA fusion gene were transformed into E. coli TG1 cells. The transformed cells were grown in YT medium. Expression was induced by 1 mM IPTG followed by shaking at 30° C. for 16 hours. Subsequently, cells were harvested by centrifugation (6000 rpm, 15 min, 4° C.) and resuspended in HisTrap buffer (20 mM sodium phosphate, 0.5 M NaCl, 40 mM imidazole, pH 7.4) in the presence of 1 mM PMSF protease inhibitor and DNAse I. Subsequently, cells were lysed by sonication and the soluble fractions were collected by centrifugation (12000 rpm, 20 min, 4° C.).

Expression Evaluation

The expression levels of Fabs and Fab-SASA fusions were estimated by Western blot. All proteins were separated by 12% SDS-PAGE under non-reducing condition and transferred to nitrocellulose membranes. After blocking, membranes were incubated with anti-His antibody, followed by Goat Anti-Mouse IgG. The signals were visualized by chemiluminescence.

BSA-Conjugated Affinity Column and BSA Magnetic Beads

For making BSA-conjugated column, BSA was prepared in the coupling buffer (0.1M NaCO₃, 0.5M NaCl, pH 8.3) and mixed with CNBr-activated Sepharose 4B (GE Heathcare). The mixture was rotated at 4° C. overnight and the excess BSA was washed away with 5 gel volumes of coupling buffer. The medium was then transferred to 0.1 M Tris (pH 8.0) to block remaining active groups, and washed by three cycles of alternating pH. The BSA-conjugated resin was packed in the protein binding buffer before use.

For preparing BSA magnetic beads, BioMagPlus Amine particles (Polysciences, Inc) were activated using 5% Glutaraldehyde. BSA was prepared in pyridine wash buffer (pH 6.0) and incubated with activated particles for 24 hours at room temperature. The reaction was quenched by 1 M Glycine (pH 8.0) and BSA coupled beads were washed with pyridine wash buffer (pH 6.0).

Purification of Fab-SASA Fusions

The purification of Fab-SASA fusions was performed on an AKTA FPLC system (GE Heathcare) using both BSA-conjugated affinity column and HisTrap HP column (GE Healthcare). Cells were resuspended in the binding buffer (20 mM sodium phosphate, 0.5 M NaCl, pH 7.4) and lysed by sonication. Cell lysates were clarified by filtration and divided into two equal portions. Half of the supernatant was applied to a 1 ml HisTrap HP column at a flow rate of 0.5 ml/min, and washed with 10 column volumes of the binding buffer. The column was further washed with binding buffer containing 80 mM imidazole to remove proteins that were non-specifically bound to the resin. Fab-SASA fusions were eluted using 200 mM imidazole. The other half of the supernatant containing Fab-SASA fusions was loaded onto a 2 ml BSA-conjugated column at a flow rate of 0.5 ml/min, and the column washed with 5 column volumes of wash buffer (20 mM sodium phosphate, pH 7.0). The column was further washed with 10 ml of 0.1 M Glycine (pH5.5) and 10 ml of 0.1 M Glycine (pH 3.5). Fab-SASA fusions were eluted with 0.1 M Glycine (pH2.2) and immediately neutralized with 1 M Tris buffer (pH9.0). The flow-through, wash fractions and eluates were analyzed by 12% SDS-PAGE.

Fab-SASA fusion was also purified by both BSA magnetic beads and Ni magnetic beads.

Cell lysates were divided into two equal portions and incubated with same amount of BSA magnetic beads and Ni magnetic beads, respectively. BSA magnetic beads were washed with 20 mM sodium phosphate, 0.5 M NaCl (pH 7.0) and eluted with 0.1 M Glycine (pH 2.2). Ni magnetic beads was washed with 20 mM sodium phosphate, 0.5 M NaCl, 5 mM imidazole (pH 7.0) and eluted with 400 mM imidazole in 20 mM sodium phosphate. Purified proteins were analyzed by 12% SDS-PAGE.

Serum Protein Concentration (Pharmacokinetics)

One of the Fabs, Fab_A07365, and its SASA fusion Fab_A07365-SASA were purified as described above. 200 μg of purified proteins were i.v. injected into mice, and their serum concentrations were measured by ELISA at 6, 24, 36, 48 and 72 hrs after injection. The measured protein concentrations in serum were plotted against time, and serum half lives of both proteins were analyzed by fitting the data to a non-compartment model using Phoenix WinNonlin 6.2 software.

Results and Discussion

The pMustKey Vector

A vector pMustKey was constructed by insertion of gene encoding the SASA tag into pSJF2 [31]. This vector was used to construct expression plasmids for protein-SASA fusions. The pMustKey vector was designed to express SASA tagged proteins (FIG. 1a). A modified pMustKey vector, pMustKey-N, was constructed as well to fuse SASA-tag to the N-terminus of a target protein (FIG. 1 b).

Expression of Target Proteins

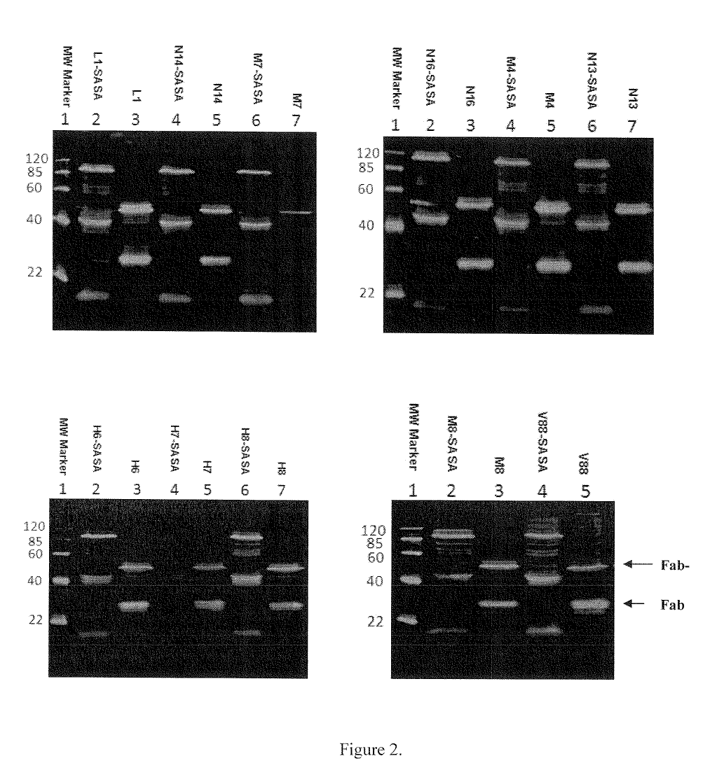

The expression system was tested with eleven Fabs. To investigate the effect of SASA-fusion on expression levels of the target proteins, the 11 Fabs were expressed in E. coli cells using both vectors with and without the SASA-tag, i.e., pMuskKey and pSJF2. The soluble fractions from cell lysates were tested using Western blot (FIG. 2). The results demonstrated that the expression levels of Fabs and Fab-SASA fusions were comparable. Hence SASA-tag does not affect the expression yield and solubility of target proteins. Interestingly, M7_Fab showed very low expression yield when expressed alone using the pSJF2 vector (FIG. 2a). In contrast, when M7_Fab was expressed as M7_Fab-SASA fusion, its expression level was increased significantly, suggesting the SASA tag has the potential to increase expression levels of low expressing proteins.

In a scale up experiment, Fab_{—}07365 and its SASA fusion Fab_A07365-SASA were both expressed with yields of approximately 5 mg/L (determined by Bradford assay), which indicated that SASA fusion could be applied in larger scale expression.

Purification of Protein-SASA Fusions

SASA tightly binds to BSA with a K_{D} of 4 pM [3], and surprisingly this strong interaction can be disrupted by low pH condition. In addition, SASA is relatively small (MW˜13 kDa), stable (Tₘ˜70° C.) and adaptable to a wide pH range [30]. These properties make SASA an ideal purification tag since it can be tightly retained by BSA-conjugated column, easily washed off from the resin, and adapted in diverse conditions which suit the target proteins.

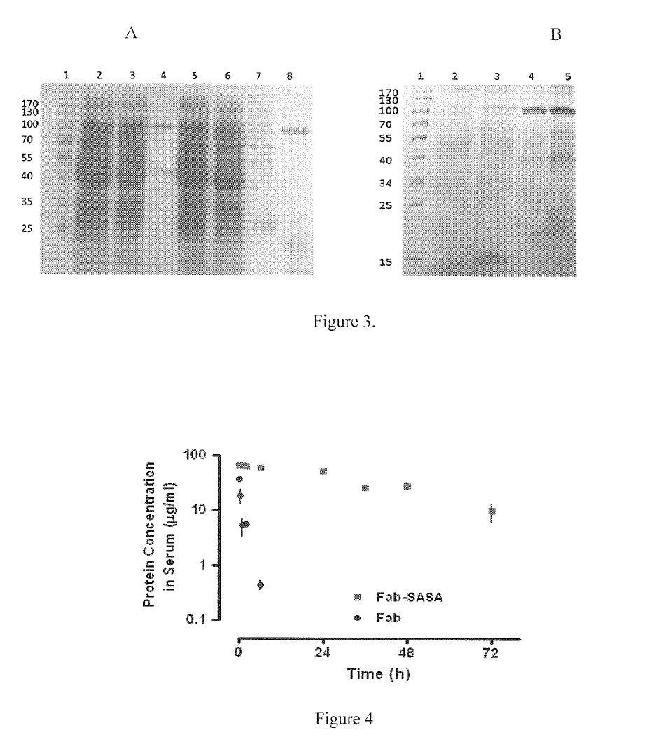

To compare the purification using the SASA tag to the common purification system, a Fab fragment expressed using the pMustKey vector was purified by immobilized metal affinity chromatography (Ni-NTA column and Ni magnetic beads) and BSA-conjugated affinity chromatography (BSA-sepharose column and BSA magnetic beads) in parallel. SDS-PAGE analysis was performed to investigate the purity of the eluted Fabs using crude and purified protein fractions (FIG. 3). The result showed that the SASA-tagged Fab was specifically purified using BSA-conjugated column with a recovery rate of 95% and >90% purity (FIG. 3A). It also indicated that the purification is more efficient by using BSA magnetic beads than by using Ni magnetic beads (FIG. 3B, 3C).

In terms of cost, BSA-conjugated column is inexpensive compared with most of the commercially available purification systems using fusion partners, such as anti-Flag, Strep and GST columns. Also, the pMustKey purification system can perform double purification for high purity demand using both His-tag and SASA-tag. In addition, this expression system is attractive for the expression and purification of non-covalently attached heterodimer proteins, since the two tags on both of the C- and N-terminus ensure that the purified proteins have both subunits.

Serum Half Life Extension

The serum half lives of a purified Fab_A07365 and Fab_A07365-SASA fusion were tested in mice and calculated by a non-compartment model (FIG. 4). Fab_A07365-SASA fusion had a serum half life of 28.7 h, representing a 28-fold increase over the Fab_A07365 which had a serum half life of 1.0 h. This direct comparison suggested that SASA significantly extended the serum half life of the SASA tagged target protein.

Extending serum half lives of protein drugs by fusion partners are generally taken place at late stage of drug development as it can be time-consuming. However, the target proteins can be tested at any stage in vivo by employing the pMustKey system since no further construction and additional formulation are required. An added advantage of the SASA tag over other fusion partners, such as albumin and Fe, is its small size, for which SASA has less effect on the function and activity of the target proteins.

Accordingly, a novel protein expression and purification system using the SASA tag and the protein-SASA fusion can be easily adapted into animal test. The target protein fused with SASA can be expressed at similar level to the non-tagged protein, and efficiently purified using BSA-conjugated column. Furthermore, SASA-tag can notably extend the serum half-life of the fused protein, which is beneficial for efficacy screening at early stage.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Jonsson A, Dogan J, Herne N, Abrahmsén L, Nygren P A. Engineering of a femtomolar affinity binding protein to human serum albumin. Protein Eng Des Sel. 2008, 21(8):515-27
[2] Kery V, Savage J R, Widjaja K, Blake B K, Conklin D R, Ho Y S, Long X, von Rechenberg M, Zarembinski T I, Boniface J J. Expression screen by enzyme-linked immunofiltration assay designed for high-throughput purification of affinity-tagged proteins. Anal Biochem. 2003, 317(2):255-8.
[3] Goh S L, Murthy N, Xu M, Frechet J M J. Cross-linked microparticles as carriers for the delivery of plasmid DNA for vaccine development. Bioconj. Chem. (2004), 15(3), 467-474.
[4] Wolber V, Maeda K, Schumann R, Brandmeier B, Wiesmtiller L, Wittinghofer A. A universal expression-purification system based on the coiled-coil interaction of myosin heavy chain. Biotechnolog 1992; 10(8):900-4.
[5] Kwong K Y, Rader C. E. coli expression and purification of Fab antibody fragments. Curr Protoc Protein Sci. 2009; Chapter 6:Unit 6.10.
[6] Hochuli E, Bannwarth W, Döbeli H, Gentz R, Stüber D. (1988). Genetic Approach to Facilitate Purification of Recombinant Proteins with a Novel Metal Chelate Adsorbent. Bio/Technology 6 (11): 1321-1325
[7] Hengen P. (1995) Purification of His-Tag fusion proteins from Escherichia coli. Trends in biochemical sciences 20 (7): 285-6.
[8] Smith D B, Johnson K S. Single-step purification of polypeptides expressed in Escherichia coli as fusions with glutathione S-transferase. Gene. 1988 15; 67(1):31-40.
[9] McCormick K L, Shetler M, Mick G J. Highly specific micromethod for the enzymatic determination of radioactive [14C]lactate. Anal Biochem. 1991; 192(2): 293-7.
[10] Schmidt T G, Skerra A. The random peptide library-assisted engineering of a C-terminal affinity peptide, useful for the detection and purification of a functional Ig Fv fragment. Protein Eng. 1993; 6(1): 109-22.
[11] di Guan C, Li P, Riggs P D, Inouye H. Vectors that facilitate the expression and purification of foreign peptides in Escherichia coli by fusion to maltose-binding protein. Gene. 1988 15; 67(1):21-30.
[12] Maina C V, Riggs P D, Grandea A G 3rd, Slatko B E, Moran L S, Tagliamonte J A, McReynolds L A, Guan C D. An Escherichia coli vector to express and purify foreign proteins by fusion to and separation from maltose-binding protein. Gene. 1988; 74(2): 365-73.
[13] Emery V C, Bishop D H. The development of multiple expression vectors for high level synthesis of eukaryotic proteins: expression of LCMV-N and AcNPV polyhedrin protein by a recombinant baculovirus. Protein Eng. 1987; 1(4):359-66.
[14] Voss S, Skerra A. Mutagenesis of a flexible loop in streptavidin leads to higher affinity for the Strep-tag II peptide and improved performance in recombinant protein purification. Protein Eng. 1997; 10(8):975-82.
[15] Skerra A, Schmidt T G. Use of the Strep-Tag and streptavidin for detection and purification of recombinant proteins. Methods Enzymol. 2000; 326:271-304.
[16] Schmidt T G, Skerra A. The Strep-tag system for one-step purification and high-affinity detection or capturing of proteins. Nat. Protoc. 2007; 2(6):1528-35.
[17] Einhauer A, Jungbauer A. J Biochem Biophys Methods. The FLAG peptide, a versatile fusion tag for the purification of recombinant proteins. 2001; 49(1-3):455-65.
[18] Thomas P. Hopp, Kathryn S. Prickett, Virginia L. Price, Randell T. Libby, Carl J. March, Douglas Pat Cerretti, David L. Urdal & Paul J. Conlon. A Short Polypeptide Marker Sequence Useful for Recombinant Protein Identification and Purification. Nat. Biotechnol. 1988; 6: 1204-1210.
[19] Lichty J J, Malecki J L, Agnew H D, Michelson-Horowitz D J, Tan S. Comparison of affinity tags for protein purification. Protein Expr Purif. 2005; 41(1):98-105.
[20] Kontermann R E. Strategies for extended serum half-life of protein therapeutics. Curr Opin Biotechnol. 2011 August; In Press.
[21] Holt L T, Basran A, Jones K, Chorlton J, Jespers L S, Brewis N D, Tomlinson I M. Protein Eng Des Sel. Anti-serum albumin domain antibodies for extending the half-lives of short lived drugs. 2008; 21(5): 283-8.
[22] Pedley R B, Boden J A, Boden R, Begent R H, Turner A, Haines A M, King D J. The potential for enhanced tumour localisation by poly(ethylene glycol) modification of anti-CEA antibody. Br J Cancer. 1994; 70(6): 1126-30.
[23] Chapman A P, Antoniw P. Spitali M, West S, Stephens S, King D J. Therapeutic antibody fragments with prolonged in vivo half-lives. Nat. Biotechnol. 1999; 17(8):780-3.
[24] Syed S, Schuyler P D, Kulczycky M. Sheffield W P Blood. Potent antithrombin activity and delayed clearance from the circulation characterize recombinant hirudin genetically fused to albumin. Blood. 1997; 89(9): 3243-52.
[25] Smith B J, Popplewell A, Athwal D, Chapman A P, Heywood S, West S M, Carrington B, Nesbitt A, Lawson A D, Antoniw P, Eddelston A, Suitters A. Prolonged in vivo residence times of antibody fragments associated with albumin. Bioconjug Chem. 2001; 12(5):750-6.
[26] Osborn B L, Olsen H S, Nardelli B, Murray J H, Zhou J X, Garcia A, Moody G, Zaritskaya L S, Sung C. Pharmacokinetic and pharmacodynamic studies of a human serum albumin-interferon-alpha fusion protein in cynomolgus monkeys. J Pharmacol Exp Ther. 2002; 303(2): 540-8.
[27] Mohler K M, Torrance D S, Smith C A, Goodwin R G, Stremler K E, Fling V P, Madani H, Widmer M B. Soluble tumor necrosis factor (TNF) receptors are effective therapeutic agents in lethal endotoxemia and function simultaneously as both TNF carriers and TNF antagonists. J. Immunol. 1993; 151(3): 1548-61.
[28] Huang C. Receptor-Fc fusion therapeutics, traps, and MIMETIBODY technology. Curr Opin Biotechnol. 2009; 20(6): 692-9.
[29] Li S, Zheng W, Kuolee R, Hirama T, Henry M, Makvandi-Nejad S, Fjällman T, Chen W, Zhang J. Pentabody-mediated antigen delivery induces antigen-specific mucosal immune response. Mol. Immunol. 2009 May; 46(8-9):1718-26.
[30] Bin M, Hirama T, Li S, Baral T, Chen Y, Yan X, Zhang J. Fast screening for expression levels, biophysical properties and affinities of large number of proteins, http://www.genscript.com/gsfiles/techfiles/FASEBA.pdf.
[31] Tanha J, Muruganandam A, Stanimirovic D. Phage Display Technology for Identifying Specific Antigens on Brain Endothelial Cells. Methods Mol. Med. 2003; 89:435-49.
[32] Chuang V T, Kragh-Hansen U, Otagiri M. Pharmaceutical strategies utilizing recombinant human serum albumin. Pharm Res. 2002; 19(5): 569-77.
[33] Peters T Jr Serum albumin. Adv Protein Chem. 1985; 37:161-245.
[34] Dennis M S, Zhang M, Meng Y G, Kadkhodayan M, Kirchhofer D, Combs D, Damico L A. Albumin binding as a general strategy for improving the pharmacokinetics of proteins. J Biol. Chem. 2002; 277(38):35035-43.
[35] Cortez-Retamozo V, Lauwereys M, Hassanzadeh Gh G, Gobert M, Conrath K, Muyldermans S, De Baetselier P, Revets H. Efficient tumor targeting by single-domain antibody fragments of camels. Int J Cancer. 200; 98(3): 456-62.
[36] WO2009127691

We claim:

1. A method of increasing the stability of a target polypeptide in a serum, the method comprising:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) administering a composition comprising the isolated fusion protein to the serum, wherein the fusion protein has a longer half-life than the target polypeptide alone in the serum.

2. The method of claim 1, further comprising:
(a) expressing the fusion protein by a recombinant cell;
(b) obtaining at least one of the lysate, periplasmic extract, and supernatant of the recombinant cell comprising the fusion protein;
(c) applying the at least one of the lysate, periplasmic extract and supernatant to a solid support to capture the fusion protein via specific binding between the sdAb in the fusion protein and the serum albumin associated with the solid support; and
(d) eluting the captured fusion protein from the solid support to obtain the isolated fusion protein.

3. The method of claim 2, wherein the recombinant cell is an Escherichia coli cell.

4. The method of claim 2, wherein the captured fusion protein is eluted at a pH of 1 to 4 or 9 to 14.

5. The method of claim 1, wherein the sdAb comprises SEQ ID NO: 2, 6 or 8.

6. The method of claim 1, wherein the serum albumin is bovine serum albumin.

7. The method of claim 1, wherein the administering step comprises administering the composition to a subject in need of a diagnosis or treatment involving the target polypeptide.

8. The method of claim 1, wherein the administering step comprises administering the composition to the serum in vivo or in vitro for identifying a diagnostic or therapeutic agent.

9. A method of increasing the stability of a target polypeptide in a composition, the method comprising:
(a) obtaining an isolated fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide; and
(b) combining the isolated fusion protein with the serum albumin in the composition, wherein the fusion protein has a longer half-life than the target polypeptide alone in the composition.

10. The method of claim 9, further comprising:
(a) expressing the fusion protein by a recombinant cell;
(b) obtaining at least one of the lysate, periplasmic extract and supernatant of the recombinant cell comprising the fusion protein;
(c) applying the at least one of the lysate, periplasmic extract and supernatant to a solid support to capture the fusion protein via specific binding between the sdAb in the fusion protein and the serum albumin associated with the solid support; and
(d) eluting the captured fusion protein from the solid support to obtain the isolated fusion protein.

11. The method of claim 10, wherein the recombinant cell is an Escherichia coli cell.

12. The method of claim 10, wherein the captured fusion protein is eluted at a pH of 1 to 4 or 9 to 14.

13. The method of claim 9, wherein the sdAb comprises SEQ ID NO: 2, 6 or 8.

14. The method of claim 9, wherein the serum albumin is bovine serum albumin.

15. The method of claim 9, further comprising administering the composition to a subject in need of a diagnosis or treatment involving the target polypeptide.

16. The method of claim 9, further comprising administering the composition to a serum in vivo or in vitro for identifying a diagnostic or therapeutic agent.

17. A method of obtaining an isolated fusion protein having increased serum stability, the method comprising:
(a) expressing the fusion protein by a recombinant cell, wherein the fusion protein comprises a fusion tag comprising the amino acid sequence of SEQ ID NO: 2, 6 or 8, the target polypeptide and an optional linker from a recombinant cell, wherein the fusion tag is fused to the carboxyl-terminus or amino-terminus of the target polypeptide, and the optional linker separates the fusion tag and the carboxyl-terminus or amino-terminus of the target polypeptide;
(b) obtaining at least one of the lysate, periplasmic extract and supernatant of the recombinant cell comprising the fusion protein;
(c) applying the at least one of the lysate, periplasmic extract and supernatant to a serum albumin affinity chromatography column; and
(d) isolating the fusion protein from the column.

18. A system for increasing the stability of a target polypeptide, comprising:
(a) a recombinant cell for expressing a fusion protein comprising a single-domain antibody (sdAb) against a serum albumin, the target polypeptide and an optional linker, wherein the sdAb is fused to the carboxyl-terminus or amino-terminus of the target polypeptide, and the optional linker separates the sdAb and the carboxyl-terminus or amino-terminus of the target polypeptide;
(b) a solid support for capturing the fusion protein via specific binding between the sdAb in the fusion protein and the serum albumin associated with the solid support;
(c) a buffer having a pH of 1 to 4 or 9 to 14 for eluting the captured fusion protein from the solid support to obtain an isolated fusion protein; and
(d) a composition comprising the serum albumin for combining with the isolated fusion protein to increase to the stability of the fusion protein.

19. The system of claim 18, wherein the recombinant cell is an Escherichia coli cell.

20. The system of claim 18, wherein the sdAb comprises SEQ ID NO: 2, 6 or 8.
